(19) United States
(12) Patent Application Publication
(10) Pub. No.: US 2010/0221350 A1
(43) Pub. Date: Sep. 2, 2010

(54) DERMATOLOGICAL COMPOSITIONS COMPRISING VITAMIN D LIPID VESICLES

(75) Inventors: Lara BAUDONNET, Fontenilles (FR); Claire Mallard, Mougins (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/647,021

(22) Filed: Dec. 24, 2009

(30) Foreign Application Priority Data
Jun. 29, 2007 (FR) 0756165
Jun. 27, 2008 (FR) PCT/FR2008/051193

(51) Int. Cl. A61K 9/50; A61K 31/59; A61P 17/06

(52) U.S. Cl. 424/490; 514/167

(57) ABSTRACT

Dermatological/pharmaceutical compositions contain lipid vesicles dispersed in a hydrophilic phase, such vesicles including at least one vitamin D compound and particularly calcitriol, and are useful for the treatment of dermatological pathologies, notably psoriasis.

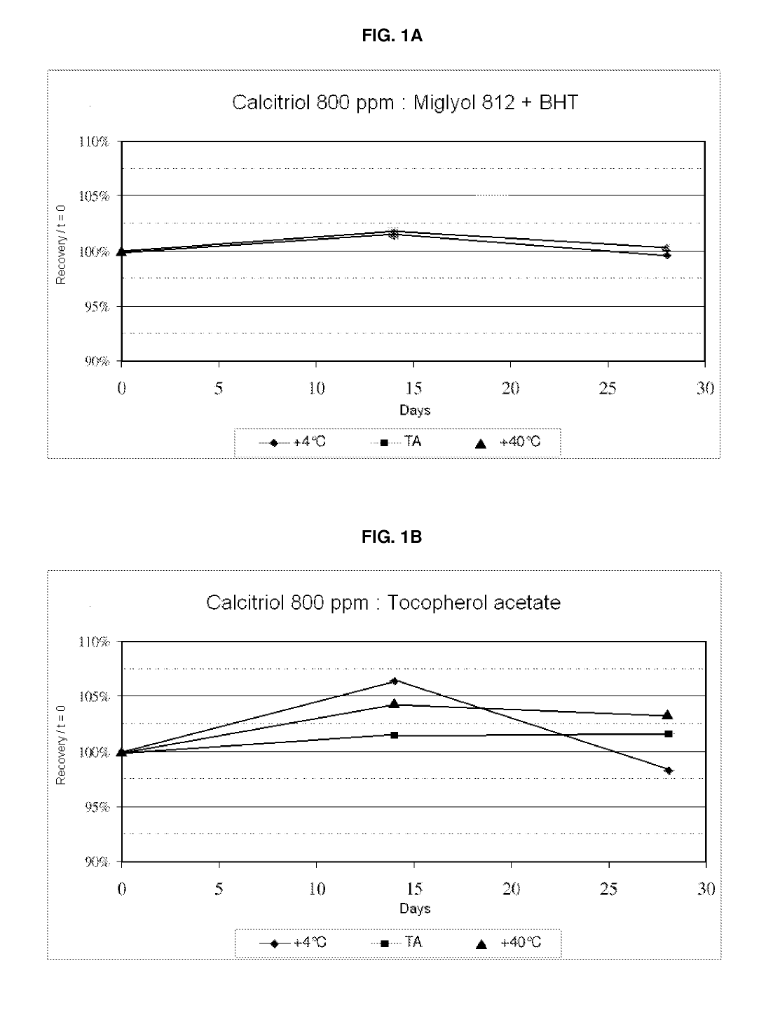

### CROSS-REFERENCE TO PRIORITY/PCT APPLICATIONS

This application claims priority under 35 U.S.C. §119 of FR 07/56165, filed Jun. 29, 2007, and is a continuation/national phase of PCT/FR 2008/051193, filed Jun. 27, 2009 and designating the United States (published in the French language on Jan. 15, 2009 as WO 2009/007622 A2; the title and abstract were also published in English), each hereby expressly incorporated by reference in its entirety and each assigned to the assignee hereof.

### BACKGROUND OF THE INVENTION

1. Technical Field of the Invention

The present invention relates to pharmaceutical compositions, in particular dermatological compositions, based on a compound of the family of the vitamin D derivatives and more particularly calcitriol, the said vitamin D compound being present within oily vesicles dispersed in an aqueous phase. This invention also relates to a process for the preparation of such compositions and to medicaments comprised thereof for the treatment of dermatological conditions, in particular psoriasis.

2. Description of Background and/or Related and/or Prior Art

Vitamin D derivatives, in particular calcitriol, are used to regulate the level of calcium in the body. The use of calcitriol in the treatment of dermatological diseases has been described in particular in U.S. Pat. No. 4,610,978 for the treatment of psoriasis.

In the prior art, the existing treatments often comprise a high percentage of petroleum jelly to promote the occlusivity and the penetration of the active principle but thus exhibit the disadvantage of being very greasy and sticky, which thus does not promote comfort and ease of application.

The other types of compositions common to the prior art comprise a high percentage of propenetrating glycol to promote the penetration of the active principle but are sticky and can give rise to problems of intolerance (“The critical role of the vehicle to therapeutic efficacy and patient compliance”, Piacquadio et al., Journal of the American Academy of Dermatology, August 1998).

### SUMMARY OF THE INVENTION

The present invention features physically and chemically stable compositions comprising at least one vitamin D compound, preferably calcitriol, for the treatment of psoriasis, such compositions exhibiting an ease of use and a cosmetic quality acceptable for application to all the areas of the body which may be affected by the pathology.

The present invention thus provides hydrophilic formulations based on a vitamin D compound, preferably calcitriol, as an alternative to the Silkis® ointment forms. Such formulations provide the patient with greater comfort and guarantee better observance of the treatment. However, vitamin D compounds and in particular calcitriol are unstable in aqueous media and are sensitive to acidic pH values.

The present invention thus provides aqueous compositions which avoid decomposition of the vitamin D compound, preferably calcitriol, while retaining good cosmetic properties. To this end, the structure of the interface from the medium for dissolution of the vitamin D derivative, preferably calcitriol, and the aqueous phase, has now been modified.

CN 1491551 describes formulations of nanocapsule type with, for active principle, ivermectin in suspension in water which are produced via polymeric emulsions, i.e., a solvent-free process employing an in situ polymerization of monomer.

Likewise, FR-2,805,761 describes lipid nanocapsules comprising phosphatidylcholines in combination with a hydrophilic cosurfactant derived from polyethylene glycol, Solutol HS 15. However, the presence of cosurfactant is necessary for the production of the nanocapsules. Furthermore, the process for the preparation of such nanocapsules takes place by phase inversion (PIT process), which entails temperature cycles in the process. Finally, the active principles are dissolved in an oil composed of medium-chain triglycerides of caprylic and capric acids, which oil is marketed under the trademark of Labrafac WL1349 by Gattefossé. These techniques are thus not applicable to vitamin D derivatives, in particular calcitriol, as they would bring about the decomposition of the active principle.

Indeed, it has now been discovered, surprisingly, that compositions comprising at least one vitamin D compound, preferably calcitriol, in the form dissolved in a dispersed oily phase make it possible to stabilize this active principle and are easy to prepare. Such compositions of the invention comprise at least one vitamin D compound, preferably calcitriol, in the form dissolved in a hydrophilic environment, do not require the inclusion of polymer or organic solvent, and guarantee the stability of the active principle. These also promote the cutaneous penetration of the active principle, which is of value in the treatment of dermatological conditions, in particular psoriasis.

One aspect of the present invention thus features compositions, in particular pharmaceutical compositions, comprising at least one vitamin D compound, preferably calcitriol, said compositions comprising, formulated into a pharmaceutically acceptable vehicle, lipid vesicles which comprise:

at least one vitamin D compound;

an oily internal phase in which the vitamin D compound is dissolved; and

a lamellar coating obtained from at least one surfactant, the said lipid vesicles being dispersed in a continuous hydrophilic phase.

### BRIEF DESCRIPTION OF THE DRAWING

The FIGURE of Drawing illustrates the results of stability and solubility tests of calcitriol in two excipients.

### DETAILED DESCRIPTION OF BEST MODE AND SPECIFIC/PREFERRED EMBODIMENTS OF THE INVENTION

The compositions according to the invention comprise from 0.00001 to 0.1% of at least one vitamin D compound by weight, with respect to the total weight of the composition, preferably from 0.0001 to 0.001%. Preferably, the compositions according to the invention comprise from 0.0001 to 0.001% of calcitriol.

“Composition of lipid vesicles” according to the invention means a colloidal lipid system comprising lipid vesicles possessing a solid or semisolid interface which are dispersed in a continuous hydrophilic phase, the said vesicles comprising an oily internal phase in which the vitamin D compound, preferably calcitriol, is dissolved, and a lamellar coating obtained from at least one nonpolymeric surfactant which forms the semisolid or solid interface from the oily internal phase and the continuous hydrophilic phase.

Vitamin D compounds means calcitriol, calcipotriol or {4-[6-ethyl-4′-(1-ethyl-1-hydroxypropyl)-2′-propylbiphenyl-3-yloxymethyl]-2-(hydroxymethyl)phenyl}methanol. Preferably, the compound used is calcitriol.

“Composition” according to the invention means the composition of lipid vesicles which is incorporated in a pharmaceutically acceptable vehicle, such as a gel, a cream gel or an emulsion, such as a cream or a lotion.

“Pharmaceutically acceptable vehicle” means an excipient or a mixture of excipients composing, with the nanoemulsion, the composition according to the invention.

In particular, the present invention features compositions comprising vesicles produced without an organic solvent.

According to the present invention, the subject composition comprise vesicles and not nanospheres. “Vesicles” means particles composed of a core (internal phase), which is liquid or semi-liquid at ambient temperature, encapsulated by a non-polymeric coating (casing or layer) which is solid at ambient temperature, in contrast to nanospheres, which are matrix particles, i.e., all of the mass of which is solid. When the nanospheres comprise a pharmaceutically acceptable active principle, the latter is finely dispersed in the solid matrix.

“Lipid vesicle” means a vesicle, the core of which comprises one or more fatty substances which is/are liquid or semi-liquid at ambient temperature and the coating (casing) of which is lipophilic and non-polymeric in nature. Specifically, the lipid vesicles according to the invention do not require any polymer and thus do not require in situ polymerization. Herein, “lipid vesicles” and “vesicles” are referred to without distinction.

“Ambient temperature” means a temperature of from 15° C. to 25° C.

The lipid vesicles according to the present invention have a mean particle size of less than 200 nm, preferably of less than 150 nm.

The lipid vesicles (referred to simply as “vesicles” subsequently) are present in the compositions according to the invention in an amount of from 10 to 30% by weight, with respect to the total weight of the composition, preferably from 10 to 20%.

The vesicles are each comprised of a core, which is liquid or semi-liquid at ambient temperature, coated with a lamellar coating obtained from at least one surfactant.

The surfactant coating (casing or layer) encapsulating the vesicles is preferably a material which is solid at ambient temperature. It is not polymeric in nature. Preferably, it has a structure of liquid crystal type.

This coating comprises one or more lipophilic surfactants; advantageously, such lipophilic surfactant(s) are selected from among anionic amphiphilic lipids. More preferably, the surfactant(s) are selected from among the family of the phospholipids, preferably from the family of the lecithins, and the lipophilic surfactant is preferably a hydrogenated lecithin, the percentage of saturated (or hydrogenated) phosphatidylcholine of which is advantageously high. “High percentage” means an amount of 70 to 99% of saturated (or hydrogenated) phosphatidylcholine, with respect to the total weight of lecithin. The phosphatidylcholines show good compatibility with the skin with a very low irritant potential.

As the lecithin according to the present invention is solid at ambient temperature, this promotes the formation of a semisolid interface in the composition.

Exemplary lecithins which can be used include natural or synthetic soya or egg lecithins having a content of hydrogenated phosphatidylcholine of greater than 70%, such as, for example, the Lipoid of grade S75-3, S100-3 or SPC-3, the Epikuron of grade 200 SH or 100H or the Phospholipon of grade 80H, 90H or 100H.

The coating of lipophilic surfactant encasing the liquid or semi-liquid core as defined above is present in an amount of from 0.1 to 10% by weight, preferably from 1 to 5% by weight, with respect to the total weight of the composition.

The coating of lipophilic surfactant, in particular of lecithin, according to the invention makes possible, by itself alone, the encapsulation of the vitamin D compound, in particular calcitriol, which prevents this active principle from coming into contact with the hydrophilic phase and thus ensures the chemical stability thereof. In particular, the composition and in particular the coating is substantially devoid of cosurfactant other than the anionic amphiphilic lipids, therefore substantially devoid of cosurfactant other than the phospholipids, and in particular substantially devoid of hydrophilic cosurfactant. Preferably, the composition does not comprise any cosurfactant other than the ionic amphiphilic lipids.

The vitamin D compound, in particular calcitriol, is thus found dissolved in the core of the vesicles. The said core, or oily internal phase, comprises at least one fatty substance which is liquid or semi-liquid at ambient temperature.

This is because the composition of the internal phase is thus essential for the stability of the active principle. Furthermore, the oily internal phase must, of course, be compatible with the active principle to be dissolved.

This oily internal phase comprises at least one oily solvent selected from among esters of tocopherols, and triglycerides and oils comprising them.

Exemplary such esters of tocopherols include vitamin E acetate, vitamin E linoleate, vitamin E nicotinate and vitamin E succinate.

Preferred triglycerides and oils comprising them include triglycerides of octanoic acid or also sunflower, maize, soya bean, cucumber, grape seed, sesame, hazelnut, apricot, macadamia, arara, castor, avocado or sweet almond oil, triglycerides of caprylic/capric acids, such as those marketed by Stearineries Dubois or those marketed under the trademarks Miglyol 810, 812 and 818 by Dynamit Nobel, jojoba oil and shea butter oil. An exemplary oily solvent according to the invention is vegetable squalane.

Preferably, the oily solvent is selected from among vitamin E acetate and triglycerides of caprylic/capric acids, such as Miglyol 812.

In addition to this/these oily solvent(s), the internal phase can also comprise one or more fatty substances which are liquid or semi-liquid at ambient temperature and which do not dissolve the active principle, such as:

2-alkylalkanols and their esters, such as: butyloctanol, hexyldecanol, octyldecanol, isostearyl alcohol, octyldodecanol, decyltetradecanol, undecylpentadecanol, dodecylhexadecanol, tetradecyloctadecanol, hexyldecyloctadecanol, tetradecyleicosanol, cetylarachidol and the mixture of isocetyl, isostearyl and isoarachidyl alcohols. All these compounds are commercially available, from Condea Vista under the trademark Isofol®, from Exxon Chemical under the trademark Exxal® or from Jarchem under the trademark Jarcol®. The compound available from Henkel under the trademark Eutanol G can be used. Exemplary esters of the said alcohols are: octyldodecyl octanoate, hexyldecyl caprylate, hexyldecyl laurate, hexyldecyl palmitate, hexyldecyl stearate and octyldodecyl meadowfoamate, which is an ester of octyldodecanol and of fatty acids derived from Limnanthes alba germ oil;

esters of fatty acids or of fatty alcohols, such as isopropyl palmitate, isostearyl neopentanoate and octyl palmitate;

esters of N-acetylated amino acid and of fatty alcohol, such as isopropyl N-lauroylsarcosinate; and

liquid ethers of fatty alcohol and of polypropylene glycol, such as the stearyl ether of polypropylene glycol including 15 propylene glycol units.

“Fatty substance” which does not dissolve the active principle means a compound in which the vitamin D compound has a solubility of less than or equal to 1%.

Such a fatty substance is present in an amount of from 90 to 99.99% by weight, with respect to the total weight of the internal phase, and in an amount of from 1 to 25% by weight, with respect to the total weight of the composition, preferably from 5 to 15%.

The continuous hydrophilic phase comprises water. This water can be demineralized water, a floral water, such as cornflower water, or a natural thermal or mineral water, for example selected from among water from Vittel, water from the Vichy basin, water from Uriage, water from La Roche-Posay, water from La Bourboule, water from Enghien-les-Bains, water from Saint Gervais-les-Bains, water from Néris-les-Bains, water from Allevard-les-Bains, water from Digne, water from Maizières, water from Neyrac-les-Bains, water from Lons-le-Saunier, water from Eaux-Bonnes, water from Rochefort, water from Saint Christau, water from Les Fumades, water from Tercis-les-Bains, water from Avéne or water from Aix-les-Bains. The water can be present at a content of from 70 to 90% by weight, with respect to the total weight of the composition, preferably from 80 to 90% by weight.

The hydrophilic phase can also comprise other hydrophilic compounds, such as preservatives or humectants.

Exemplary preservatives include those of parabens or of phenoxyethanol.

Exemplary humectants include those of glycerol.

The hydrophilic phase can also comprise active principles which are insensitive to the presence of water, these active principles being useful in the treatment of dermatological pathologies.

Such active principles are selected in particular from corticosteroids. According to an advantageous embodiment of the invention, the corticosteroid is selected from the group consisting of betamethasone, clobetasol, clobetasone, desoximetasone, diflucortolone, diflorasone, fluocinonide, flumethasone, fluocinolone, fluticasone, fluprednidene, halcinonide, hydrocortisone, mometasone, triamcinolone and their pharmaceutically acceptable esters and acetonides and mixtures thereof.

Examples of esters or acetonides are those selected from the group consisting of the 17-valerate, the 17-propionate, the 17,21-dipropionate, the acetonide, the acetonide, 21-N-benzoyl-2-methyl-β-alaninate, the acetonide, 21-(3,3-dimethylbutyrate) and the 17-butyrate.

Preferably, the hydrophilic phase can comprise clobetasol 17-propionate (or “clobetasol propionate”).

Advantageously, the amount of corticosteroid in the form dissolved in the hydrophilic phase of the composition of the invention ranges from 0.00005 to 3% by weight, with respect to the total weight of the composition, preferably from 0.0001 to 1% by weight and more particularly from 0.001 to 0.1% by weight.

Alternatively, the corticosteroids present in the composition as described above can be present within the oily vesicles according to the present invention. In this case, they are encountered in the oily internal phase which comprises the dissolved vitamin D derivative.

The composition, in particular the hydrophilic phase, can also comprise a gelling agent. This gelling agent is preferably a cellulose derivative selected from among semisynthetic cellulose gelling agents, such as methylcellulose, ethylcellulose, hydroxypropylmethylcellulose, hydroxyethylcellulose, carboxymethylcellulose, hydroxymethylcellulose and hydroxypropylcellulose, whether alone or as a mixture. Preferably, hydroxypropylmethylcellulose or hydroxyethylcellulose is employed. These compounds are marketed in particular by Dow Chemical under the trademark of Methocel® (for example: Methocel® E4M) or by Hercules under the trademark of Natrosol® (for example: Natrosol® 250 HHX). The gelling agent can also be selected from among natural gums, such as gum tragacanth, guar gum, gum acacia or gum arabic, starch and its derivatives, copolymers of acrylic acid and of methyl methacrylate, carboxyvinyl polymers, polyvinylpyrrolidones and their derivatives, polyvinyl alcohols, sodium alginate, pectin, dextrin or chitosan, whether alone or as a mixture. The gelling agent can also be selected from among the compound Sepigel 305, composed of a polyacrylamide/C₁₃-C₁₄ isoparaffin/laureth-7 mixture, or Simulgel 600PHA, namely sodium acryloyldimethyltaurate copolymer/isohexadecane/polysorbate 80, these two products being marketed by Seppic.

The gelling agent is included in particular at a concentration of from 0.1 to 10% by weight, preferably from 0.1 to 2% by weight.

In a preferred embodiment according to the invention, the lipid vesicle composition as described above is incorporated in a pharmaceutically acceptable vehicle, such as a gel, a cream gel or an emulsion, such as a cream or a lotion.

“Pharmaceutically acceptable vehicle” means an excipient or a mixture of excipients comprising a composition according to the invention.

The present invention thus features compositions, in particular pharmaceutical compositions, comprising the nanoemulsion containing the lipid nanovesicles defined above in a pharmaceutically acceptable vehicle, such as a gel, a cream gel or an emulsion, such as a cream or a lotion.

When the pharmaceutically acceptable vehicle is a gel, the nanoemulsion is dispersed in a hydrophilic phase which comprises at least one gelling agent. This gelling agent is preferably a cellulose derivative selected from among semisynthetic cellulose gelling agents, such as methylcellulose, ethylcellulose, hydroxypropylmethylcellulose, hydroxyethylcellulose, carboxymethylcellulose, hydroxymethylcellulose and hydroxypropylcellulose, whether alone or as a mixture. Preferably, hydroxypropylmethylcellulose or hydroxyethylcellulose is used. These compounds are marketed in particular by Dow Chemical under the trademark of Methocel® (for example: Methocel® E4M) or by Hercules under the trademark of Natrosol® (for example: Natrosol® 250 HHX). The gelling agent can also be selected from among natural gums, such as gum tragacanth, guar gum, gum acacia or gum arabic, starch and its derivatives, copolymers of acrylic acid and of methyl methacrylate, carboxyvinyl polymers, polyvinylpyrrolidones and their derivatives, polyvinyl alcohols, biopolymers, such as sodium alginate, pectin, dextrin, chitosan or sodium hyaluronate, and their derivatives, whether alone or as a mixture. The gelling agent can also be selected from among the compound Sepigel 305, composed of a polyacrylamide/C₁₃-C₁₄ isoparaffin/laureth-7 mixture, or Simulgel 600PHA, namely sodium acryloyldimethyltaurate copolymer/isohexadecane/polysorbate 80, these two products being marketed by Seppic.

The gelling agent is included in particular at a concentration of from 0.1 to 10% by weight, preferably from 0.1 to 2% by weight, with respect to the total weight of the composition.

When the pharmaceutically acceptable vehicle is a cream gel, the nanoemulsion is dispersed in a vehicle comprising a hydrophilic phase and a fatty phase.

When the pharmaceutically acceptable vehicle is an emulsion, the nanoemulsion is dispersed in a vehicle comprising a hydrophilic phase, a fatty phase and at least one surfactant or emulsifier.

In the case of the pharmaceutical vehicles in the cream-gel or emulsion form, the composition according to the invention thus comprises a fatty phase. This fatty phase can comprise, for example, vegetable, mineral, animal or synthetic oils, silicone oils and mixtures thereof.

Examples of mineral oils include liquid paraffins with different viscosities, such as Primol 352®, Marcol 82® or Marcol 152®, which are marketed by Esso.

Exemplary vegetable oils include sweet almond oil, palm oil, soya bean oil, sesame oil or sunflower oil.

Exemplary animal oils include lanolin, squalene, fish oil or mink oil, with, as derivative, the squalane marketed under the trademark Cosbiol® by Laserson.

Exemplary synthetic oils include an ester, such as cetearyl isononanoate, for example the product marketed under the trademark of Cetiol SN PH® by Cognis France, isopropyl palmitate, such as the product marketed under the trademark of Crodamol IPP® by Croda, diisopropyl adipate, marketed under the trademark of Crodamol DA by Croda, or caprylic/capric triglyceride, such as Miglyol 812®, marketed by Hüls/Univar.

Exemplary volatile or nonvolatile silicone oils are the dimethicones, such as the products marketed under the trademark of Q7-9120® Silicone Fluid with a viscosity of 20 cSt to 12 500 cSt or the product marketed under the trademark ST-Cyclomethicone-5 NF® by Dow Corning.

It is also possible to employ solid fatty substances, such as natural or synthetic waxes, fatty acids, such as stearic acid, fatty alcohols, such as Speziol C18 Pharma, marketed by Cognis, and texturing agents of tribehenate type, such as Compritol 888, marketed by Gattefossé, or hydrogenated castor oils, such as Cutina HR, marketed by Cognis. In this case, one skilled in the art will adjust the heating temperature for the preparation depending on the presence or absence of these solids.

Preferably, when the vehicle of the composition according to the invention is an emulsion, the emulsion is in the form of an oil-in-water (O/W) emulsion. The emulsion thus comprises at least one emulsifier.

The preferred concentrations of emulsifiers are from 0.001 to 20% by weight, with respect to the total weight of the composition. More preferably, the concentration ranges from 1 to 15% by weight and preferably from 3 to 11% by weight, with respect to the total weight of the composition.

The emulsifying power of the emulsifiers is closely related to the polarity of the molecule. This polarity is defined by the HLB (Hydrophilic/Lipophilic Balance).

A high HLB indicates that the hydrophilic fraction is predominant, and, conversely, a low HLB indicates that the lipophilic part is predominant. For example, HLB values of greater than approximately 10 correspond to hydrophilic surfactants.

The emulsifiers can be categorized, according to their structure, under the generic terms “ionic” (anionic, cationic or amphoteric) or “nonionic”. Nonionic emulsifiers are emulsifiers which do not dissociate into ions in water and are thus insensitive to variations in pH.

Nonionic emulsifiers are particularly well suited for the preparation of emulsions of oil-in-water type which are a subject-matter of the present invention. Thus, the emulsifying system which is a component of the emulsion of the invention comprises at least one nonionic emulsifier possessing a hydrophilic predominant fraction, that is to say exhibiting a high HLB of greater than approximately 10.

Non-limiting examples of nonionic emulsifiers exhibiting a high HLB include the sorbitan esters, such as POE(20) sorbitan monooleate, marketed under the trademark of Tween 80® (HLB=15); POE(20) sorbitan monostearate, marketed under the trademark of Tween 60® (HLB=14.9); ethers of fatty alcohols, such as POE (21) stearyl ether (HLB=15.5), marketed under the trademark of Brij 721® by Uniqema, or ceteareth-20, marketed under the trademark of Eumulgin B2® (HLB of 15.5) by Cognis, polyoxyethylene glycol esters, such as the glyceryl stearate and PEG 100 stearate marketed under the trademark of Arlacel 165 FL® (HLB=11) by Uniqema or the PEG 6 stearate and PEG 32 stearate marketed under the trademark of Tefose 1500® (HLB=10) by Gattefossé, or sugar esters with a high HLB, such as PEG 20 methyl glucose sesquistearate, marketed under the trademarks of Glucamate SSE200 (HLB=15) by Amerchol, and sucrose laurate, marketed under the trademark of Surfhope C-1216® (HLB=16), and sucrose stearate, marketed under the trademark of Surfhope C-1811® (HLB=11) by Gattefossé. Preferably, the said nonionic emulsifiers with a high HLB exhibit an HLB of from 10 and 18.

Non-limiting examples of nonionic emulsifiers with a low HLB (lipophilic) include the sorbitan esters, such as sorbitan monostearate (HLB=4.7), marketed under the trademark of Span 60® by Uniqema, glycerol esters, such as glycerol monostearate, marketed under the trademark of Cutina GMSVPH(HLB=3.8) by Cognis, polyethylene glycol esters, such as PEG-6 isostearate, marketed under the trademark of Olepal Isostéarique® (HLB=8) by Gattefossé, or sugar esters with a low HLB, such as methyl glucose sesquistearate, marketed under the trademark of Glucate SS®(HLB=6) by Amerchol, and sucrose dilaurate, marketed under the trademark of Surfhope C-1205 (HLB=5), and sucrose tristearate, marketed under the trademark of Surfhope C-1803® (HLB=3) by Gattefossé.

Preferably, the said nonionic emulsifiers exhibiting a low HLB exhibit an HLB of less than 10.

The nonionic emulsifiers can be used alone or as a mixture of two or more of them to form the emulsifying system which is a component of the emulsion of the invention.

Use will preferably be made, as emulsifying system, of one or more “nonionic emulsifier with a high HLB”/“nonionic emulsifier with a low HLB” pairs; the system can in particular be a nonionic emulsifying system comprising at least one nonionic emulsifier exhibiting an HLB of greater than approximately 10 and at least one nonionic surfactant exhibiting an HLB of less than approximately 10.

The ratio of each of the two emulsifiers forming the abovementioned pair is generally determined by the calculation of the HLB required for the fatty phase used.

Preferred emulsifiers include hydrophilic emulsifiers of the following types: glyceryl stearate & PEG-100 stearate, marketed under the trademark Arlacel 165FL® by Uniqema; PEG 6 stearate and PEG 32 stearate, marketed under the trademark of Tefose 1500® by Gattefossé, PEG 20 methyl glucose sesquistearate, marketed under the trademark of Glucamate SSE 20® by Amerchol, polyoxyethylene (21) stearyl ether, marketed under the trademark Brij 721® by Uniqema, and ceteareth-20, marketed under the trademark of Eumulgin B2PH® by Cognis; or of lipophilic emulsifiers of the following type: methyl glucose sesquistearate, such as Glucate SS®, marketed by Amerchol.

The emulsifiers are amphiphilic compounds which have a hydrophobic part having an affinity for the oil and a hydrophilic part having an affinity for the water, thus creating a link from the two phases. Ionic or nonionic emulsifiers thus stabilize O/W emulsions by being adsorbed at the interface and by forming lamellar layers of liquid crystals.

The oil-in-water emulsion according to the invention also comprises a hydrophilic phase or aqueous phase.

“Hydrophilic phase” which constitutes the pharmaceutically acceptable vehicle, alone or in an emulsion, means any hydrophilic phase as defined above in the present invention.

The compositions according to the invention can additionally comprise, in the nanoemulsion or the pharmaceutically acceptable vehicle, additives or combinations of additives, such as:

preservatives;

propenetrating agents;

stabilizing agents;

humectants;

moisture regulators;

pH regulators;

osmotic pressure modifiers;

chelating agents;

UV-A and UV-B screening agents;

and antioxidants.

Exemplary propenetrating agents which can be employed according to the invention include the glycols, such as, for example, propylene glycol, glycol ethers, N-methyl-2-pyrrolidone or dimethyl sulfoxide.

Of course, one skilled in the art will take care to select the optional compound or compounds to be added to these compositions in such a way that the advantageous properties intrinsically attached to the present invention are not or not substantially detrimentally affected by the envisioned addition.

These additives can be present in the composition at from 0 to 20% by weight, with respect to the total weight of the composition.

The pharmaceutical compositions which comprise the composition of lipid vesicles in a pharmaceutically acceptable vehicle, according to the invention are useful for the treatment of the skin and can be administered topically, parenterally or orally, whether regime or regimen. Preferably, the composition is administered topically. “Topically” means an application to the skin or mucous membranes.

Orally, the pharmaceutical composition can be provided in the liquid or pasty form and more particularly in the form of hard gelatin capsules, sugar-coated tablets or syrups.

Parenterally, the composition can be provided in the form of suspensions for infusion or for injection.

Topically, the composition can be provided in the liquid or pasty form and more particularly in the form of creams, milks, salves, impregnated pads, syndets, wipes, gels, sprays, foams, lotions, sticks, shampoos or washing bases.

The compositions according to the invention comprise, in a pharmaceutically acceptable vehicle, by weight, with respect to the total weight of the composition:

a) from 0.1 to 5% of surfactant selected from among anionic amphiphilic lipids, preferably lecithin;

b) from 1 to 50% of fatty substance which is liquid or semi-liquid at ambient temperature, preferably esters of tocopherols, such as vitamin E acetate, or triglycerides of caprylic/capric acids; preferably, from 10 to 25%;

c) from 0.00001 to 0.1% of at least one vitamin D compound, preferably calcitriol; and

d) from 0 to 2% of gelling agent, preferably a cellulose derivative.

Another aspect of the invention is a process for the preparation of the compositions comprising at least one vitamin D compound, preferably calcitriol. This process involves a High Pressure Homogenizer (HPH). In particular, the process according to the invention does not employ Phase Inversion Temperature (PIT) (used in particular in FR-2,805,761 and FR-2,840,531) and thus does not require cycle(s) in which the temperature is raised and lowered. This is because the process according to the invention takes place in the HPH under cold conditions; the HPH thus does not require successive heating and cooling and is not temperature-regulated.

The process employed in the present invention comprises the following stages:

(i) Dissolving the Vitamin D Compound in a Fatty Substance which is Liquid or Semi-Liquid at Ambient Temperature, to Obtain the Oily Phase.

Preferably, calcitriol is dissolved in the fatty substance, for example vitamin E acetate or triglycerides of caprylic/capric acids.

(ii) Mixing the Hydrophilic Compounds, to Obtain the Hydrophilic Phase.

In particular, the preservative(s) is (are) mixed with water.

(iii) Dispersing the Lipophilic Surfactant in the Oily Phase Obtained in (i) or in the Hydrophilic Phase Obtained in (ii).

The lipophilic surfactant, in particular lecithin, for example Phospholipon 90H or Lipoid S75-3, is dispersed in the oily phase.

In a preferred embodiment, the two phases (lipophilic, without the vitamin D compound, and hydrophilic) are preferably heated separately to a temperature preferably of greater than 50° C. The lipophilic phase is heated to approximately 60° C. to facilitate the dispersing of the surfactant (stage (iii)). In this case, the lipophilic phase is allowed to cool before the incorporation of the solution of vitamin D derivative (stage (i)), to prevent any decomposition of the latter related to the temperature at which the vesicles are processed.

(iv) Mixing the Oily and Hydrophilic Phases Obtained on Conclusion of Stage (iii).

Once the two phases are at temperature, approximately 50° C., the latter are mixed with stirring. Once this prehomogenization has been carried out, the emulsion is introduced into the High Pressure Homogenizer (HPH).

(v) Introducing the Mixture Obtained in (iv) into the High Pressure Homogenizer, to Obtain the Composition.

The use of a High Pressure Homogenizer makes it necessary to set the number of passes through the homogenization chamber and the homogenization pressure. The homogenization process is then applied:

minimum 500 bar up to 1,000 bar of homogenization pressure in the homogenization chamber,

from 5 and 10 passes through the homogenization chamber.

During the passes through the homogenization chamber, the composition is not heated and the HPH system is not temperature-controlled.

(vi) Incorporating the Composition of Lipid Vesicles in the Pharmaceutically Acceptable Vehicle.

In the case of a composition in the form of a gel, the stage of gelling the composition takes place at the end of manufacturing after the various passes through the HPH, during the cooling of the composition.

The gelling agent is then added with stirring sufficient for homogeneous dispersing, during the cooling of the composition. The stirring is maintained for the time necessary to bring the stage of gelling of the system to completion.

In the case of a cream gel or of an emulsion, the cream gel or the emulsion is produced beforehand. The composition of lipid vesicles is then incorporated in the finalized vehicle.

The process can be adapted by one skilled in the art according to the various ingredients used to maintain the stability of the lipid vesicles in the final composition.

The compositions according to the invention can be formulated as medicament.

In particular, another aspect of the invention is the formulation of the subject compositions into medicaments useful to treat dermatological conditions, in particular human dermatological conditions.

The administration of the compositions according to the invention is more particularly useful for the treatment of psoriasis. This is because the compositions according to the invention are suitable for use in the treatment of dermatological conditions, in particular human dermatological conditions, and preferably in the treatment of psoriasis.

To further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Preformulation Study on Calcitriol

Preliminary preformulation studies were carried out to direct the choice of the lipid core (oily internal phase) of the lipid vesicles. The results of tests of stability and of solubility of calcitriol in two excipients are presented in the graphs below. The objective of these solubility and stability studies on calcitriol is to find the oil which will make possible good solubility of calcitriol for the proposals for internal phase used in this concept and good stability of calcitriol in this excipient.

Results:

Calcitriol is stable at 800 ppm in Miglyol 812 (caprylic/capric triglyceride: MTC) and tocopherol acetate at the three temperatures (4° C., ambient temperature and 40° C.) for 1 month.

On conclusion of this study, calcitriol mother solutions were prepared in Miglyol 812 and tocopherol acetate.

### Example 2

### Formulations of Compositions of Lipid Vesicles Before Incorporation in the Pharmaceutically Acceptable Vehicle

| | Formulation | Formulation | Formulation | Formulation |
|---|---|---|---|---|
| Constituent | 1 | 2 | 3 | 4 |
| Calcitriol 0.3% | 5% | 5% | 5% | 5% |
| Miglyol 812 | | | | |
| solution | | | | |
| Miglyol 812 | 8.8% | 8.8% | 8.8% | 8.8% |
| Phospholipon | | 2% | | 2% |
| 90H | | | | |
| Lipoid S75-3 | 2% | | 2% | |
| Nipagin N M | 0.2% | 0.2% | 0.2% | 0.2% |
| Purified water | q.s. | q.s. | q.s. | q.s. |
| | for 100% | for 100% | for 100% | for 100% |

| Constituent | Chemical name | Function |
|---|---|---|
| Purified water | Purified water | Vehicle |
| Phospholipon 90H | Hydrogenated | Lipid interface |
| | phosphatidylcholine | |
| Lipoid S75-3 | Hydrogenated | Lipid interface |
| | phosphatidylcholine | |
| Nipagin M | Methyl para- | Antibacterial |
| | hydroxybenzoate | preservative |
| Calcitriol 0.3% | Caprylic/capric | Active principle |
| Miglyol solution | triglyceride | mother solution |

### Example 3

### Example of Formulations According to the Invention in the Form of a Gel

| | Formulation | Formulation | Formulation | Formulation |
|---|---|---|---|---|
| Constituent | 1′ | 2′ | 3′ | 4′ |
| Calcitriol 0.3% | 5% | 5% | 5% | 5% |
| Miglyol 812 | | | | |
| solution | | | | |
| Miglyol 812 | 8.8% | 8.8% | 8.8% | 8.8% |
| Phospholipon | | 2% | | 2% |
| 90H | | | | |
| Lipoid S75-3 | 2% | | 2% | |
| Nipagin N M | 0.2% | 0.2% | 0.2% | 0.2% |
| Gelling agent - | | | 0.5% | 0.5% |
| for example | | | | |
| cellulose | | | | |
| derivative | | | | |
| Purified water | q.s. | q.s. | q.s. | q.s. |
| | for 100% | for 100% | for 100% | for 100% |

### Example 4

### Process for the Preparation of the Formulations of Examples 2 and 3

The process employed in this invention uses a High Pressure Homogenizer (HPH).

Manufacturing Stages:

Dissolution of the Calcitriol:

The calcitriol is dissolved in the oily phase, in this instance in Miglyol 812, to produce the mother solution comprising 0.3% of calcitriol.

2. Preparation of the Hydrophilic Phase:

The preservative is dissolved in the water.

3. Dispersion of the Hydrogenated Phosphatidylcholine:

The hydrogenated phosphatidylcholine employed is dispersed in the oily phase according to the content of phosphatidylcholine.

The oily phase is heated to approximately 75° C. to ensure that the phosphatidylcholine is dispersed in this phase.

4. Introduction of the Calcitriol Mother Solution:

Before incorporating the calcitriol mother solution in the oily phase, the temperature of this phase must be approximately 50° C., to prevent decomposition of the calcitriol related to the temperature at which the lipid vesicles are processed.

5. Placing the Hydrophilic Phase at Temperature:

The hydrophilic phase is heated to approximately 50° C.

6. Mixing the Phases:

Once the two phases are at temperature, the latter are mixed with stirring (Turrax homogenization, 2 minutes at 8000 rpm).

Once this prehomogenization has been carried out, the emulsion is introduced into the HPH.

7. High Pressure Homogenization:

The use of a High Pressure Homogenizer makes it necessary to set the number of passes through the homogenization chamber and the homogenization pressure.

The homogenization process is then applied:

minimum 500 bar up to 1000 bar of homogenization pressure in the homogenization chamber,

from 5 and 10 passes through the homogenization chamber.

During the passes through the homogenization chamber, the nanoemulsion is not heated and the HPH system is not temperature-controlled.

8. Gelling:

When it takes place in the case of example of formulations as presented in Example 3, the stage of gelling of the composition takes place at the end of preparation after the various passes through the HPH, during the cooling of the composition.

The gelling agent is then added with stirring sufficient for homogeneous dispersing, during the cooling of the composition, or the gelling agent is hydrated in a portion of the water of our system then diluted at the end of manufacturing.

Stirring is maintained for the time necessary to bring the stage of gelling the system to completion.

### Example 4

### Stability Studies on the Formulations of Examples 2 and 3

Physical Stability of the Lipid Vesicles:

Particle size analysis: Zetasizer: Nano series Nano ZS (Malvern)

Two dilutions are used to carry out the particle size analyses:

1d: 10 μl of the composition in 15 ml of filtered distilled water

2d: 1 ml of 1d in 5 ml of distilled water

Formulation 2: (Phospholipon 90H):

| | | | | T 1 | T 5 | T 6 |
|---|---|---|---|---|---|---|
| | Conditions | T 0 | T 15 d | month | months | months |
| Size (nm) | +4° C. | / | 213 ± 4.8% | 227 ± 5.7% | 224 ± 4.4% | 214 ± 4.57% |
| | AT | 183.33 ± 2.2% | 213.4 ± 3.9% | 189.4 ± 1.4% | 182 ± 2.9% | 213 ± 5.63% |
| | +40° C. | / | 195.3 ± 1.9% | 190.9 ± 1.8% | 190.6 ± 5.44% | 206 ± 2.54% |
| PDI | +4° C. | / | 0.199 | 0.130 | 0.154 | 0.157 |
| | AT | 0.08 | 0.092 | 0.066 | 0.07 | 0.154 |
| | +40° C. | / | 0.071 | 0.064 | 0.087 | 0.07 |
| pH | +4° C. | / | | | 6.97 | 6.51 |
| | AT | 6.78 | | | 6.36 | 5.75 |
| | +40° C. | / | | | 6.08 | 5.25 |

The particle size is stable over time, 6 months, whatever the temperature conditions, 4° C., ambient temperature and 40° C.

We have thus succeeded in obtaining a system which is physically stable without the addition of a cosurfactant by using solely a hydrogenated phosphatidylcholine and without using solvent.

2—Chemical Stability of Calcitriol in the Oily Core of the Lipid Vesicles of Examples 2 and 3:

| Formula | 2 | | | 3 | | |
|---|---|---|---|---|---|---|
| T 0 | 178 μg/g | | | 183 μg/g | | |
| | 4° C. | AT | 40° C. | 4° C. | AT | 40° C. |
| T 1 month (% p/r T 0) | 102% | 102% | 94% | 99% | 102% | 100% |
| T 5 months (% p/r T 0) | 100% | 100% | 85% | 100% | 102% | 96% |

The lipid vesicles comprising a semi-rigid interface based on phosphatidylcholine increase the stability of calcitriol in water, whatever the stabilizing temperature, 4° C., ambient temperature and 40° C.

The gelling of the hydrophilic phase shows an improvement in the stability of the calcitriol in the lipid vesicles at 40° C.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A dermatological/pharmaceutical composition including at least one vitamin D compound, which comprises lipid vesicles dispersed in a continuous hydrophilic phase, the said lipid vesicles comprising an oily internal phase in which the at least one vitamin D compound is dissolved and a lamellar coating obtained from at least one surfactant selected from among anionic amphiphilic lipids.

2. The dermatological/pharmaceutical composition as defined by claim 1, formulated into a pharmaceutically acceptable vehicle therefor.

3. The dermatological/pharmaceutical composition as defined by claim 1, said at least one vitamin D compound comprising calcitriol.

4. The dermatological/pharmaceutical composition as defined by claim 1, said at least one surfactant comprising a phospholipid.

5. The dermatological/pharmaceutical composition as defined by claim 4, characterized in that it is substantially devoid of cosurfactant other than the phospholipid(s).

6. The dermatological/pharmaceutical composition as defined by claim 1, wherein the oily internal phase of the vesicles comprises a fatty substance which is liquid or semi-liquid at ambient temperature.

7. The dermatological/pharmaceutical composition as defined by claim 1, wherein the oily internal phase of the vesicles comprises at least one oily solvent selected from among esters of tocopherols and triglycerides and oils comprising thereof.

8. The dermatological/pharmaceutical composition as defined by claim 6, wherein the fatty substance is present in an amount ranging from 90 to 99.99% by weight, with respect to the total weight of the internal phase.

9. The dermatological/pharmaceutical composition as defined by claim 1, wherein the at least one surfactant is selected from among lecithins comprising from 70 to 99% by weight of hydrogenated phosphatidylcholine.

10. The dermatological/pharmaceutical composition as defined by claim 1, wherein the at least one surfactant is present in an amount ranging from 0.1 to 10% by weight, with respect to the total weight of the composition

11. The dermatological/pharmaceutical composition as defined by claim 1, comprising at least one gelling agent.

12. The dermatological/pharmaceutical composition as defined by claim 11, wherein the at least one gelling agent comprises a cellulose derivative selected from among methylcellulose, ethylcellulose, hydroxypropylmethylcellulose, hydroxyethylcellulose, carboxymethylcellulose, hydroxymethylcellulose and hydroxypropylcellulose, and mixtures thereof.

13. The dermatological/pharmaceutical composition as defined by claim 1, comprising at least one corticosteroid.

14. The dermatological/pharmaceutical composition as defined by claim 1, which comprises, in water, by weight, with respect to the total weight of the composition:
a) from 0.1 to 5% of at least one surfactant selected from among anionic amphiphilic lipids;
b) from 1 to 20% of at least one fatty substance which is liquid or semi-liquid at ambient temperature;
c) from 0.00001 to 0.1% of at least one vitamin D compound; and
d) from 0 to 2% of at least one gelling agent.

15. The dermatological/pharmaceutical composition as defined by claim 14, which comprises, in water, by weight, with respect to the total weight of the composition:
a) from 0.1 to 5% of lecithin;
b) from 1 to 20% of esters of tocopherols or of triglycerides of caprylic/capric acids;
c) from 0.00001 to 0.1% of calcitriol; and
d) up to 2% of cellulose derivative.

16. The dermatological/pharmaceutical composition as defined by claim 1, formulated for topical administration.

17. The dermatological/pharmaceutical composition as defined by claim 1, formulated as a medicament.

18. A regime or regimen for the treatment of a dermatological condition, comprising administering to an individual in need of such treatment, a thus effective amount of the dermatological/pharmaceutical composition as defined by claim 1.

19. A regime or regimen for the treatment of psoriasis, comprising administering to an individual in need of such treatment, a thus effective amount of the dermatological/pharmaceutical composition as defined by claim 1.

20. A process for the formulation of a dermatological/pharmaceutical composition as defined by claim 1, which comprises the following stages:
(i) dissolving the at least one vitamin D compound in at least one fatty substance which is liquid or semi-liquid at ambient temperature, to obtain the oily phase;
(ii) mixing hydrophilic compounds, to obtain the hydrophilic phase;
(iii) dispersing the at least one lipophilic surfactant in the oily phase obtained in (i) or in the hydrophilic phase obtained in (ii);
(iv) mixing the oily and hydrophilic phases obtained on conclusion of stage (iii);
(v) introducing and homogenizing the mixture obtained in (iv) into a High Pressure Homogenizer, to provide said composition.

21. The process as defined by claim 20, which comprises a stage (vi) of addition of at least one gelling agent into the composition obtained in (v).

22. A dermatological/pharmaceutical composition including at least one vitamin D compound, which comprises lipid vesicles dispersed in a continuous hydrophilic phase, the said lipid vesicles comprising an oily internal phase in which the at least one vitamin D compound is dissolved and a lamellar coating obtained from at least one lipid surfactant.
